(19) United States
(12) Patent Application Publication
(10) Pub. No.: US 2010/0298437 A1
(43) Pub. Date: Nov. 25, 2010

(54) USE OF CITRULLINE FOR TREATING UNDERNUTRITION CONDITIONS

(75) Inventors: Christophe Moinard, Bourg La Reine (FR); Marion Jourdan, Paris (FR); Stephane Walrand, Aydat (FR); Luc Cynober, Sceaux (FR)

Correspondence Address: YOUNG & THOMPSON, 209 Madison Street, Suite 500, Alexandria, VA, 22314, US

(73) Assignees: UNIVERSITE RENE DESCARTES-PARIS 5, Paris Cedex 06 (FR); BIOCODEX, Gentilly (FR)

(21) Appl. No.: 12/445,820

(22) PCT Filed: Aug. 29, 2007
(86) PCT No.: PCT/FR07/01407

§ 371 Date: Jul. 14, 2009

(30) Foreign Application Priority Data
Oct. 17, 2006 (FR) 06/09077

(51) Int. Cl. A61K 31/198; A61P 3/00; A61P 35/00

(52) U.S. Cl. 514/565; 514/564

(57) ABSTRACT

The invention relates to the use of L-citrulline (I) or of one of its pharmaceutically acceptable salts in the preparation of a drug for the treatment of states or undernutrition as linked to a lowering of protein synthesis within the framework of pathologies which do not result from an intestinal insufficiency.

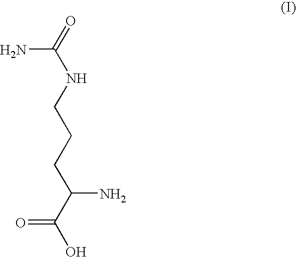

This invention relates to the use of citrulline in the preparation of a drug for the treatment of certain undernutrition conditions.

Many catabolic states are characterized by an undernutrition, with a lowering of muscle protein anabolism. This lowering of proteosynthesis participates in the amyotrophy which is observed in many catabolic states, and fosters the establishment of a cachexia. Now it is well known that the latter is a factor which worsens morbidity and mortality rates (Schneider S M, Veyres P, Pivot X, Soummer A M, Jambou P, Filippi J et al. Malnutrition is an independent factor associated with nosocomial infections. Br J Nutr 2004; 92:105-111).

The inventors have already shown that the enteral administration of L-citrulline to rats which suffer from undernutrition conditions due to an intestinal insufficiency allowed these rats to take on weight again (French patent application FR 03 08349 published under N^{o} FR 2 857 262).

The invention which is described in that patent application results from the demonstration of the fact that the enteral administration of L-citrulline to rats who suffer from undernutrition conditions due to an intestinal insufficiency, allowed these rats to take on weight again.

The experiments described in French patent application N^{o} FR 03 08349 were carried out in vivo on rats having undergone a severe resection of the small intestine. This resection resulted in a lowering of nutriment absorption, and therefore in an intestinal insufficiency, leading to undernutrition conditions.

The uses of citrulline as derived from this demonstration are those of the treatment:
of the short bowel syndrome following an intestinal resection,
of the celiac disease,
of chronic inflammatory diseases of the intestine, such as Crohn's disease, and ulcerous rectocolitis,
of age-linked intestinal insufficiency,
of irradiation-linked intestinal insufficiency,
   i.e. of the treatment of pathologies which are all linked to an intestinal insufficiency, whatever the cause of the latter.

Within the framework of former studies on undernourished ageing rats, the inventors more particularly observed an intestinal insufficiency, also called age-linked intestinal insufficiency, essentially resulting from a splanchnic sequestration mechanism of amino acids which no longer circulate in the periphery (Curis E, Nicolis I, Moinard C, Osowska S, Zerrouk N, Benazeth S et al. Almost all about citrulline in mammals. Amino Acids 2005; 29:177-205).

As citrulline is not retained by the splanchnic area the inventors suggested that citrulline might be a vector of nitrogen in the periphery.

They thus demonstrated in in vivo experiments that bringing citrulline to undernourished aged rats could restore a protein synthesis level which is equivalent to the baseline protein synthesis level among healthy aged rats.

These experiments would allow one to suggest that citrulline may be used within the framework of the treatment of age-linked intestinal insufficiency (as mentioned in French patent application N^{o} FR 03 08349).

The inventors have studied within the framework of this invention the direct effect of citrulline on the muscle by carrying out in vitro experiments adding citrulline on isolated muscles of healthy or undernourished adult rats.

Indeed there are no data in the literature to suggest that the muscle has any capacity to transform citrulline into a substance which would be active on protein synthesis, nor that citrulline has a direct action on the muscle protein synthesis.

Therefore the inventors wanted to study the fate of citrulline in the muscle in order to explain the metabolic mechanism leading to an effect on the stimulation of protein synthesis.

Thus the inventors have shown:
that the muscle of healthy or undernourished adult rats has no capacity to metabolize citrulline, considering that one does not find any in vitro liberation of amino acids which would be metabolically linked to citrulline in the test medium,
and that adding citrulline to undernourished adult rat muscles increases the protein synthesis of these muscles by up to 30%, whereas no effect is observed on protein synthesis by adding in vitro citrulline on the muscles of healthy adult rats.

Therefore the inventors have shown for the first time that, in an unexpected manner, citrulline is not metabolized in the muscle and has a direct action on the muscle protein synthesis, which is independent from any intestinal insufficiency which would be linked to a digestive pathology or to any modification of the digestive metabolism.

Thus one of the aims of the invention is to provide a means of treating states of undernutrition, and more particularly cachexia when this is linked to a lowering in the protein synthesis within the framework of pathologies which are not linked to a renal insufficiency.

Another aim of the invention is to provide a means for increasing an abnormally low intramuscular protein synthesis level in patients who are in a state of undernutrition linked to a lowering of protein synthesis within the framework of pathologies which do not result from intestinal insufficiency.

The invention relates to the use of L-citrulline (I)

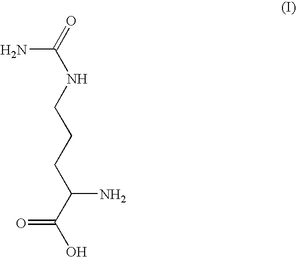

or of one of its pharmaceutically acceptable salts in the preparation of a drug for the treatment of states of undernutrition which are linked to a lowering of the protein synthesis within the framework of pathologies which do not result from an intestinal insufficiency.

Within the scope of this invention the term L-citrulline is used to denote the product which is found on the market, notably that which is provided by Sigma, or the product which is naturally obtained from plants, notably from watermelon (Citrullus lanatus) in the form of juice, pulp or extract.

“Intestinal insufficiency” is used to denote a pathological state of the intestine, notably the small intestine, wherein the absorption of nutriments is reduced when compared with normal, the lowering of the absorption of nutriments being linked to a lowering in the number and/or functionality of intestinal cells which are able to ensure this absorption, and wherein this lowering of the number and/or the functionality of intestinal cells is itself due either to a physical elimination of these cells (notably by surgery or by the use of rays), or to a pathological dysfunction of these cells.

The invention particularly relates to the use of L-citrulline in the preparation of a drug for increasing an abnormally low intramuscular protein synthesis level among patients in a state of undernutrition which is linked to a lowering of protein synthesis within the framework of pathologies which do not result from an intestinal insufficiency.

The invention more particularly relates to the use, as above mentioned, of L-citrulline in the preparation of a drug for the treatment of the following disorders or pathologies:
proteino-energetic malnutrition linked to an insufficient intake,
cancer, except intestinal cancer when resulting in an intestinal insufficiency,
muscle denervation,
chemotherapies, excluding those which act at the intestinal level,
diabetes,
obesity,
weightlessness,
immobilized limb after fracture,
regulated surgery, excluding intestinal digestive surgery, and
dystrophy.

Disorders or pathologies which may be treated within the framework of this invention are illustrated in an article by Couet et al. (Couet C., Attaix D. Le muscle. In: Leverve X, Cosnes J, Erny P, Hasselmann M, editors. Traitéde nutrition artificielle de l'adulte. Paris: Mariette Guéna, 1998: 261-274).

The invention also relates to a method for the therapeutic treatment of the above-mentioned disorders and pathologies, comprising administering to a patient an effective dose of citrulline or of one of its salts.

The invention relates to L-citrulline for the treatment of the above-mentioned disorders and pathologies.

More particularly the invention relates to the use of L-citrulline in the preparation of a drug for the treatment of patients which suffer from undernutrition conditions which is linked to a lowering of the protein synthesis level within the frame of pathologies which do not result from an intestinal insufficiency.

Therefore the invention relates to L-citrulline in the treatment of patients which suffer from a state of undernutrition which is linked to a lowering of the protein synthesis level within the frame of pathologies which do not result from an intestinal insufficiency.

According to another embodiment the invention relates to the above-mentioned use of L-citrulline in the preparation of a pharmaceutical composition which comprises, as an active substance, L-citrulline or one of its pharmaceutically acceptable salts in association with a pharmaceutically acceptable excipient.

Notably the term “pharmaceutically acceptable salt” is used to denote citrulline salts such as citrulline malate, citrulline α-ketoglutarate, citrulline citrate or citrulline α-ketoisocaproate.

Pharmaceutically acceptable excipients will appear as self-evident to any art specialist.

The invention notably relates to the above-mentioned use of L-citrulline in the preparation of a pharmaceutical composition, characterized in that the L-citrulline unit dose is between ca. 2 g-ca. 20 g, notably ca. 10 g, for a dosage regimen of between ca. 0.1 g/kg/day-ca. 0.5 g/kg/day, notably ca. 0.25 g/kg/day.

More particularly the invention relates to the above-mentioned use of L-citrulline in the preparation of a pharmaceutical composition which may be in the form of a dry composition or as an aqueous solution.

More particularly still, the invention relates to the above-mentioned use of L-citrulline in the preparation of a pharmaceutical composition which may be found in a form which may be administered orally, subcutaneously, enterally or parenterally.

Enteral administration notably corresponds to the administration through a stomach tube, a naso-gastric probe or a naso-intestinal probe, by gastrotomy or jejunostomy, and parenteral administration notably corresponds to the administration by way of central, peripheral or subcutaneous intravenous perfusion.

More particularly the invention relates to the above-mentioned use of L-citrulline in the preparation of a pharmaceutical composition which also comprises one or several other compounds for the treatment of undernutrition-linked cachexia, such as leucine, glutamine, arginine, ornithine and their various applicable salts such as α-ketoglutarate or α-ketoisocaproate, whether isolated or in a nutritional mixture for parenteral nutrition, or a mixture for enteral nutrition, or a mixture for oral nutrition.

According to another embodiment the invention relates to a pharmaceutical composition, characterized in that it comprises, as an active substance, L-citrulline, or one of its pharmaceutically acceptable salts, in association with at least another compound for the treatment of cachexia when linked to undernutrition, such as leucine, glutamine, arginine, ornithine and their various acceptable salts such as α-ketoglutarate or α-ketoisocaproate, whether isolated or in a nutritional mixture for parenteral nutrition, or a mixture for enteral nutrition, or a mixture for oral nutrition, and with a pharmaceutically acceptable excipient.

According to another embodiment the invention relates to products which comprise:
L-citrulline or one of its pharmaceutically acceptable salts,
and at least another compound for the treatment of undernutrition-linked cachexia, such as leucine, glutamine, arginine, ornithine and their various acceptable salts such as α-ketoglutarate or α-ketoisocaproate, whether isolated or within a nutritional mixture for parenteral nutrition, or a mixture for enteral nutrition, or a mixture for oral nutrition, as combination products for a simultaneous, separated or delayed use, within the framework of the treatment of intestinal insufficiency.

The invention is illustrated with the following Examples 1-2 and FIGS. 1-3.

### DESCRIPTION OF FIGURES

FIG. 1A represents the Fractional Protein Synthesis (FSR) of epitrochlearis as obtained from healthy rats (left columns) or undernourished rats (right columns), which have been incubated in the presence of citrulline (black columns) or in the absence of citrulline (control—white columns) as measured according to the procedure of Example 1. Results are expressed in %/hour.

FIG. 1B represents the Fractional Protein Synthesis (FSR) of epitrochlearis as obtained from healthy rats (left columns) or undernourished rats (right columns), which have been incubated in the presence of citrulline (black columns) or in the absence of citrulline (control—white columns) as measured according to the procedure of Example 1. Results are expressed in %/control.

FIG. 2A represents the Western Blot after 1 hr development illustrating the activation of P70S6kinase on muscles as obtained from undernourished aged rats, as measured according to the procedure of Example 2.

FIG. 2B is a graphic representation of the activation of P70S6kinase on muscles as obtained from undernourished aged rats as measured according to the procedure which is described in Example 2. AL: control group; R: group undergoing dietary restriction; R+AANE: group undergoing dietary restriction, then following a standard diet which is enriched in non essential amino acids; R+CITR: group undergoing dietary restriction, then following a standard diet which is enriched in citrulline (5 g/kg/d). Statistic tests: ANOVA+PLSD Fisher's test: *versus AL, p<0.05; ¤ versus R, p<0.05.

### EXAMPLE 1

### Regulation of the Muscle Protein Synthesis by Citrulline as Measured in Vitro On an Isolated Perifused Muscle

### 1.1. Materials and Methods

Treatment of animals: Male Sprague-Dawley rats (Charles River Laboratoires, L'Arbresles, France) aged 3 months (n=20) are placed in individual cages in a thermostated atmosphere (23°±1° C.), and subjected to a 12 hr light/dark cycle (dark between 8 a.m.-8 p.m.).

Acclimatization of the rats is carried out during 2 weeks, during which the spontaneous food consumption is measured. The rats are fed a standard diet (A04, UAR, Villemoisson-sur-Orge, France) containing 17% proteins, 3% lipids, 59% carbohydrates and 21% water, fibers, vitamins and minerals. The average dietary intake during this period is 28 g/day for adult rats.

At the end of the acclimatization period, the rats are randomized in 2 groups: a control group made up of rats which are fed ad libitum (AL), and a group which is subjected to dietary restrictions during the same period: the rats are fed at a rate of 50% of spontaneous ingesta during 6 weeks with a 5% protein diet (Walrand S, Chambon-Savanovitch C, Felgines C, Chassagne J, Raul F, Normand B et al. Aging: a barrier to renutrition? Nutritional and immunologic evidence in rats. Am J Clin Nutr 2000; 72:816-824).

Incubated isolated muscles. Muscles were incubated according to a method which had been used previously (Minet-Quinard R, Moinard C, Villie F, Vasson M P, Cynober L. Metabolic pathways implicated in the kinetic impairment of muscle glutamine homeostasis in adult and old glucocorticoid-treated rats. Am J Physiol Endocrinol Metab 2004; 287:E671-E676). Epitrochlearis is used because it is the most suitable for this type of study. After dissection, the epitrochlearis are incubated in 3 mL Krebs-Ringer buffer (119 mM NaCl; 4.8 mM KCl; 1.25 mM MgSO₄; 25 mM NaHCO₃; 1.24 mM NaHPO₄; 1.0 mM CaCl₂; 2 mM HEPES, pH 7.4), also containing glucose (8 mM), insulin (0.01 U/ml) and bovine serum albumin (BSA) (0.1% p/v). The muscles are pre-incubated during 30 minutes at 37° C. with 95% O₂: 5% CO₂. The muscles are then transferred into a tube containing 3 mL incubation medium (with ¹³C-phenylalanine (1 mM) with or without 2.5 mM citrulline), and are incubated during 2 hours. At the end of the incubation, the muscles are collected and kept at −80° C. until the incorporation of ¹³C-phenylalanine is measured by mass spectrometry in order to determine the Fractional Protein Synthesis (FSR) (Guillet C, Boirie Y, Walrand S. An integrative approach to in-vivo protein synthesis measurement: from whole tissue to specific proteins. Curr Opin Clin Nutr Metab Care 2004; 7:531-538). Moreover amino acids are titrated in the incubation medium by ion exchange chromatography.

### 1.2. Results

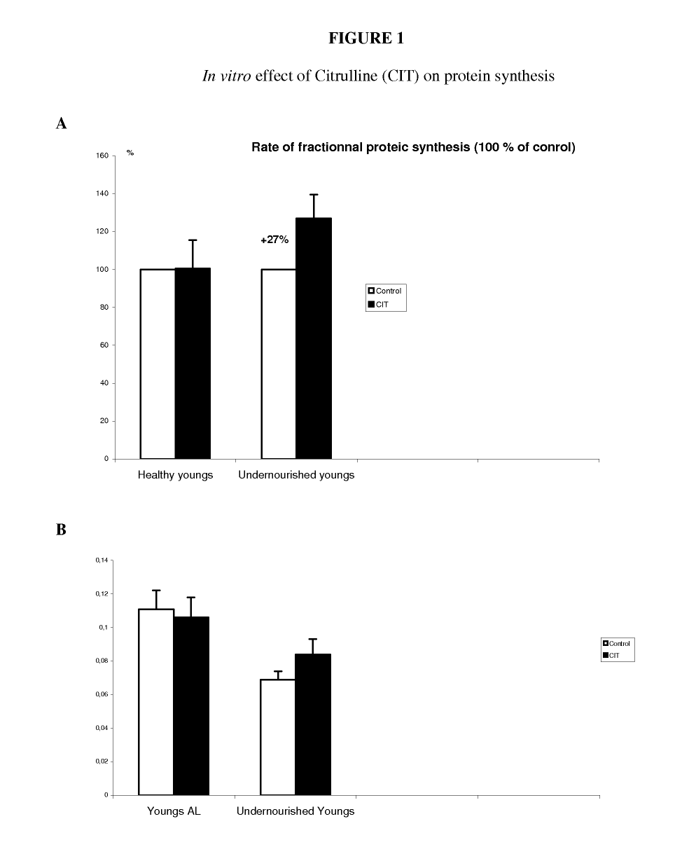

They are given in FIG. 1.

Citrulline is not metabolized by the muscle because no amino acid which is metabolically linked to citrulline is released in the incubation medium (results not shown).

The results show that, according to literature data, undernutrition lowers the muscle protein synthesis level in adult rats (white column, healthy young rats as compared with white column, undernourished young rats, FIG. 1A).

The results of the above-mentioned experiments show that the administration of L-citrulline allows one to increase the muscle protein synthesis in undernourished rats with a lowering of the muscle protein synthesis level (+27% FIG. 1A, comparison between white column and black column of young undernourished rats). This work, having been carried out ex vivo on incubated isolated muscles, allows one to show that L-citrulline has a direct action at the muscle level. This result is surprising and was totally unpredictable in view of literature data, because only leucine (an essential amino acid) possesses such a property (Crozier S J, Kimball S R, Emmert S W, Anthony J C, Jefferson L S. Oral leucine administration stimulates protein synthesis in rat skeletal muscle. J Nutr 2005; 135:376-382). Now L-citrulline is an amino acid whose structure is very different from that of leucine; it does not enter into the composition of proteins, and is almost absent from ordinary diets.

### EXAMPLE 2

### In Vivo Study Demonstrating a Direct Action on Protein Synthesis Through the Activation of the mTOR Pathway

### 2.1. Materials and Methods

Treatment of animals: Forty male Sprague-Dawley rats aged 19 months (Charles River, L'Arbresle, France) were used. During an acclimatization period of 2 weeks the animals were fed ad libitum with a standard diet (UARA04, Usine d'Alimentation Rationnelle, Villemoisson-sur-Orge). Measurement of the spontaneous ingesta was carried out regularly. After this period 30 rats were subjected to a 50% dietary restriction during 12 weeks. Sacrificing 10 rats, used as a control group (group R), was carried out at the end of the dietary restriction period. The other 20 animals were again fed during one week before being sacrificed. Isonitrogen and isocaloric diets were either a standard diet enriched with citrulline 5 g/kg/d (n=10, group R+CIT), or a standard diet enriched with non essential amino acids (Ala, Gly, His, Asp, Ser in an equimolar ratio) (n=10, group R+AANE). Ten rats make up the control group (A.L); they are fed ad libitum all along the study before being sacrificed. Then the rats are sacrificed. The tibialis muscles are taken and quickly frozen in liquid nitrogen, then stored at −80° C. until analysis.

Extraction of proteins and titration: The muscles are ground in liquid nitrogen with a mortar. The thus obtained powder is weighed, and then taken again in 10 volumes of a solubilization buffer containing a cocktail of phosphatase and protease inhibitors. The samples are placed at least 1 hour on a wheel in a freezing chamber. After centrifugation during 30 minutes at +4° C. and 13,000 g, the supernatant is divided between aliquot parts and kept at −80° C.

Proteins are titrated with the bicinchoninic acid method.

An aliquot part of each sample is denaturated in a bain-marie at 100° C. during 5 minutes in a denaturation buffer containing β-mercaptoethanol (5%) and a Laemmli buffer (Laemmli Sample Buffer).

Analysis of the activation of the mTOR (mammalian Target of Rapamycin) is carried out by Western Blot, showing the phosphorylated forms of the various targets of mTOR. Protein integrity is checked by polyacrylamide gel electrophoresis (SDS-PAGE), followed by a Coomassie Blue staining.

Western Blot technique: 20 μg denaturated proteins are left on a 10% and 12% polyacrylamide gel, respectively, for the study of p70^{S6K} and rpS6 (phosphorylated or total forms).

Migration of proteins by electrophoresis: The migration is carried out at 20 mA during 2 hours in a 1× (Tris 25 mM, Glycine 192 mM, SDS 0.1%) migration buffer.

Transfer of the proteins onto a nitrocellulose membrane: The gels are then transferred onto a nitrocellulose membrane. To this effect they are each placed in a small box. Transfer is made in the cold in a 1× transfer buffer (Tris 25 mM, Glycine 192 mM, SDS 0.01%, absolute ethanol 20%), at 120 mA during a minimum of 1 hr 30 nm. The quality of the transfer is visualized by poppy red staining.

Immunodetection: The membranes are pre-incubated during 1 hour in an appropriate buffer [Tris Buffer (Tris Buffer Saline Tween 1× (TBST)]: Tris 1 mM, NaCl 15 mM, Tween 20 0.5%, pH 8; 1% powder skimmed milk, 4% BSA) in order to saturate non specific sites. They are then incubated during one night at 4° C. in a hybridization buffer according to the protein form to be studied. In order to study the phosphorylated form the TBST 1× buffer, 1% skimmed milk, 4% BSA, is mixed with the primary antibody. For the study of the total form, the TBSA buffer (TBST 1×, 5% skimmed milk) is used for mixing with the primary antibody (P70 S6kinase total #9202 dilué au 1/125^{e}; anti Phospho-P70 S6kinase (Thr389) #9234 1/250 diluted; rpS6 (5G10) total #2217 1/2000 diluted; Phospho-rpS6 (Ser240/244) #2215 1/2000 diluted). After several rinsings in a TBST 1× solution, 3 washing steps of 20 minutes are carried out in the same solution. The membranes are then incubated during ¾ hr with the second antibody, as coupled with the peroxydase. Again they are twice rinsed in TBST 1×, then washed 3 times minimum during 20 minutes in the same solution. Development on radiographic film is carried out in a dark room with the ECL kit.

### 2.2. Results

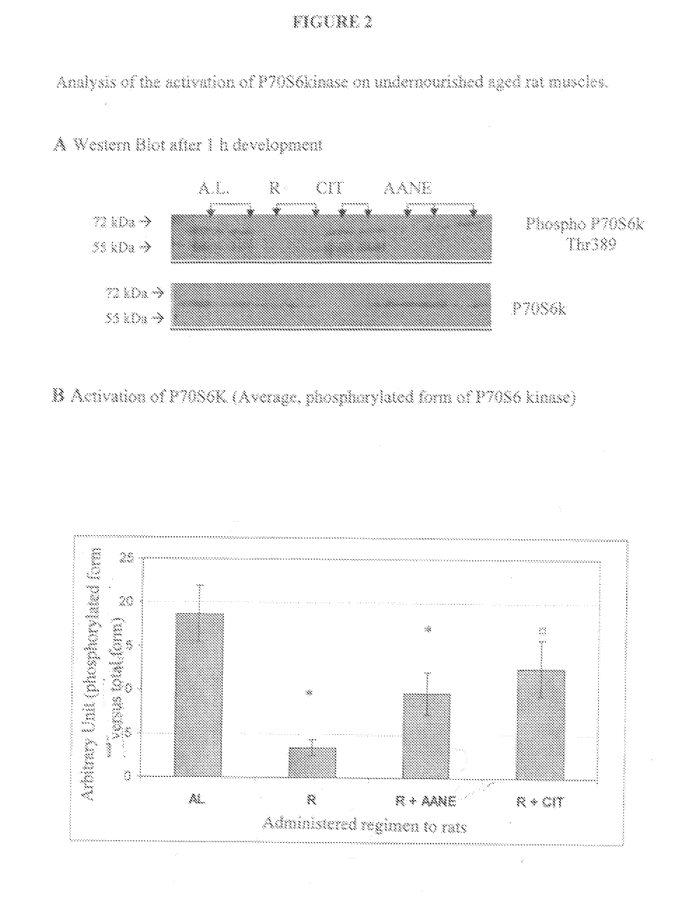

They are given in FIG. 2.

The results show that dietary restriction (group R) significantly lowers the activity of P70S6 kinase (−83% as compared with the group A.L. controls).

A diet which is supplemented with non essential amino acids (group R+AANE) restores part of this activity, but in a non significant manner, said activity remaining significantly below that observed with controls (−45% as compared with the group A.L. controls).

On the contrary a diet which is supplemented with citrulline at a rate of 5 g/kg/d (group R+CIT) significantly restores this activity (+417% as compared with group R), said activity not being significantly different from that which is observed in the control group (group A.L.).

Thus these results show that citrulline has a direct activity on protein synthesis through the activation of the mTOR system.

1-8. (canceled)

9. A method for treatment of undernutrition conditions linked to a lowering of the protein synthesis within the framework of pathologies which do not result from an intestinal insufficiency comprising the administration of L-citrulline (I) 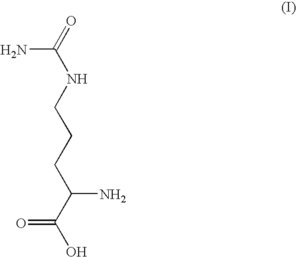
or of one of its pharmaceutically acceptable salts.

10. Method according to claim 9, intended to increase intramuscular protein synthesis when abnormally low among patients who are under undernutrition conditions which is linked to a lowering of protein synthesis within the framework of pathologies which do not result from an intestinal insufficiency.

11. Method according to claim 9 wherein disorders or pathologies as chosen from among the group comprising:
protein energy malnutrition as linked to an intake deficiency,
cancers, except intestinal cancer leading to an intestinal insufficiency,
muscle denervation,
chemotherapies, except those which have an action at the intestinal level,
diabetes,
obesity,
weightlessness,
limbs which are immobilized after a fracture,
regulated surgery, except intestinal digestive surgery, and
dystrophy.

12. Method according to claim 10 wherein disorders or pathologies as chosen from among the group comprising:
protein energy malnutrition as linked to an intake deficiency,
cancers, except intestinal cancer leading to an intestinal insufficiency,
muscle denervation,
chemotherapies, except those which have an action at the intestinal level,
diabetes,
obesity,
weightlessness,
limbs which are immobilized after a fracture,
regulated surgery, except intestinal digestive surgery, and
dystrophy.

13. Method according to claim 9, characterized in that the active substance is L-citrulline or one of its pharmaceutically acceptable salts, in association with a pharmaceutically acceptable excipient.

14. Method according to claim 10, characterized in that the active substance is L-citrulline or one of its pharmaceutically acceptable salts, in association with a pharmaceutically acceptable excipient.

15. Method according to claim 9, characterized in that the unit dose of L-citrulline is between ca. 2 g-ca. 20 g, notably ca. 10 g, for a dosage regimen of ca. 0.1 g/kg/day-ca. 0.5 g/kg/day, notably ca. 0.25 g/kg/day.

16. Method according to claim 9, characterized in that the pharmaceutical composition is obtained in the form of a dry composition or of an aqueous solution.

17. Method according to claim 9, characterized in that the pharmaceutical composition may be found in a form which may be administered orally, subcutaneously, enterally or parenterally.

18. Method according to claim 9, characterized in that the pharmaceutical composition also comprises one or several other compounds for the treatment of cachexia as linked to undernutrition, such as leucine, glutamine, arginine, ornithine and their various acceptable salts such as α-ketoglutarate or α-ketoisocaproate, whether isolated or within a nutritional mixture for parenteral nutrition, or a mixture for enteral nutrition, or a mixture for oral nutrition.

19. Method according to claim 10, characterized in that the unit dose of L-citrulline is between ca. 2 g-ca. 20 g, notably ca. 10 g, for a dosage regimen of ca. 0.1 g/kg/day-ca. 0.5 g/kg/day, notably ca. 0.25 g/kg/day.

20. Method according to claim 10, characterized in that the pharmaceutical composition is obtained in the form of a dry composition or of an aqueous solution.

21. Method according to claim 10, characterized in that the pharmaceutical composition may be found in a form which may be administered orally, subcutaneously, enterally or parenterally.

22. Method according to claim 10, characterized in that the pharmaceutical composition also comprises one or several other compounds for the treatment of cachexia as linked to undernutrition, such as leucine, glutamine, arginine, ornithine and their various acceptable salts such as α-ketoglutarate or α-ketoisocaproate, whether isolated or within a nutritional mixture for parenteral nutrition, or a mixture for enteral nutrition, or a mixture for oral nutrition.
